(19) United States
(12) Patent Application Publication
(10) Pub. No.: US 2021/0196147 A1
(43) Pub. Date: Jul. 1, 2021

(54) USE OF CO VALUES IN SMOKING CESSATION

(71) Applicant: Carrot Inc., Reedwood City, CA (US)

(72) Inventors: Allen JAMESON, Sunnyvale, CA (US); David S. UTLEY, Redwood City, CA (US); Matthew HALL, London (GB)

(73) Assignee: Carrot Inc., Reedwood City, CA (US)

(21) Appl. No.: 17/132,513

(22) Filed: Dec. 23, 2020

Related U.S. Application Data
(60) Provisional application No. 62/955,555, filed on Dec. 31, 2019.

(51) Int. Cl. A61B 5/08; A61B 5/00

(52) U.S. Cl. CPC A61B 5/082; A61B 5/486; A61B 5/7425

(57) ABSTRACT

Use of co values in smoking cessation are described where an apparatus may be configured to encourage smoking cessation in a user. The apparatus may include a sampling unit which receives a sample breath and determines a level of exhaled carbon monoxide from the sample breath. The sampling unit may be programmed to initiate a first timer for tracking a cumulative time upon a determination that the level of exhaled carbon monoxide is below a predetermined threshold. The sampling unit may also be programmed to initiate a second timer for tracking a countdown time upon a determination that the level of exhaled carbon monoxide is above the predetermined threshold, wherein the countdown time corresponds to a second length of time until the carbon monoxide level within the body of the user is expected to dissipate below the predetermined threshold.

### CROSS-REFERENCE TO RELATED APPLICATIONS

This application claims the benefit of priority to U.S. Prov. App. 62/955,555 filed Dec. 31, 2019, which is incorporated herein by reference in its entirety.

### FIELD OF THE INVENTION

The present invention relates to apparatus and methods for receiving and detecting biological parameters from breath samples. In particular, the present invention relates to apparatus and methods of use for receiving and detecting biological parameters from breath samples exhaled from a subject and into a portable breath sensor apparatus for educating and motivating users to decrease and quit their use of cigarettes.

### BACKGROUND OF THE INVENTION

The health problems associated with tobacco smoking are well known. Cigarette smoke contains nicotine as well as many other chemical compounds and additives. Tobacco smoke exposes an individual to carbon monoxide (CO) as well as these other compounds, many of which are carcinogenic and toxic to the smoker and those around the smoker. The presence and level of CO in the exhaled breath of the smoker can provide a marker for identifying the overall smoking behavior of that individual as well as provide a marker for their overall exposure to the other toxic compounds.

In order to sample the exhaled breath, a portable breath sensor which is readily carried by the user and which is unobtrusive is desirable. While this portable breath sensor can measure the exhaled carbon monoxide (eCO) values of users, it may not be immediately intuitive to all users how to use this data since it may not be a widely understood metric.

As cigarette smoke contains CO in concentrations that can exceed 1% by volume (e.g., 10,000 PPM), the CO that is inhaled with cigarette smoke enters into the lungs and passes into the bloodstream where the CO binds to hemoglobin (Hb) which has a stronger affinity for CO than for oxygen (approximately 350 times stronger). Because of this affinity, CO outcompetes oxygen for binding sites on Hb and stays bound to Hb for longer periods of time. The CO gradually disassociates from Hb and passes back into the lungs where it is subsequently exhaled. Hence, the concentration of CO in exhaled breath is proportional to the concentration of Hb carrying CO. For instance, if 5% of a person's Hb is carrying CO instead of oxygen, the person's exhaled breath will contain approximately 30 PPM of CO. As a result of the CO exhalation dynamics, the concentration of CO-bound Hb, and the resulting concentration of eCO, the CO exhibits a half-life decay with a half-life of approximately 3 to 4 hours within the user's body.

Accordingly, there remains a need for a breath sensor which is able to sample and accurately detect parameters from exhaled breath and to use the eCO results to educate and motivate users to decrease and ultimately quit their use of cigarettes.

### SUMMARY OF THE INVENTION

For users who are interested in quitting smoking, the eCO values obtained from the user may be used to educate and motivate the user to quit by helping the user visualize and internalize the link between their smoking behavior and their measured eCO value. By helping the user understand this link, they may come to understand how they can change or reduce their eCO values by targeting an eCO value within a preferred range, e.g., 0 to 6 PPM characterized as a “green” range, which is a low level of CO detected from the user's exhaled breath as indicative of the user having refrained from smoking for a period of time prior to sampling their eCO. This in turn may increase the user's satisfaction with their breath sensor as well as a quit program provided to the user.

Examples of breath sampling devices and methods for determining and quantifying eCO levels from a user are described in further detail in various patents, e.g., U.S. Pat. Nos. 9,861,126; 10,206,572; 10,306,922; and 10,335,032, each of which is incorporated herein by reference in its entirety and for any purpose.

The eCO values obtained from the user may be used to educate and motivate the user to quit by helping the user visualize and internalize the link between their smoking behavior and their measured eCO value. With the eCO value quantified, the half-life of CO within the user's body may be used to predict the amount of time for the CO to dissipate to a desirable level, e.g., 6 PPM or less which is the typical CO level of a non-smoking individual, and this information may be used to provide motivational content or guidance to the user. For instance, the targeted level of 6 PPM or less may be presented to the user as a “green” level where the estimated time until the CO is dissipated so that the detected amount is within the green level can be considered “Time To Green” (TTG).

The half-life of CO within a user's body may be used to monitor their response to cigarettes as a proxy for blood volume or hemoglobin measurement. The half-life value may also be used as an indicator for potential blood health issues such as low hemoglobin values.

Presented with this information, the user may be motivated to set a goal to reach the green level which represents the typical CO level of a non-smoking individual. Each time the user takes a measurement of eCO with the unit (or personal electronic device or computer) may calculate the approximate TTG until the user will reach the green level.

Rather than using a standarized half-life value for CO dissipation within the body, an individualized model may be created for a specific user to generate a personalized half-life which accounts for a user's activity metrics such as amount of sleep, activity levels, etc. The model may also consider the number of cigarettes smoked as self-reported by the user for the personalized model in addition to the incorporated CO which may already be present within the user's body.

In one variation, the unit may use a standardized CO half-life time (e.g., around 4 hours) in determining the TTG of the user. This standardized CO half-life time may be based upon a conservative population-average in which the CO half-life time (e.g., the time for a CO level to dissipate to half within a body) is an average value obtained from a population. To account for variables such as sensor variability, sensor noise, natural breath readings, etc. the start and end values may be compensated as appropriate, e.g., to target a value of 4 PPM instead of a value of 6 PPM which is at the end of the green range.

In another variation, an individualized model may be created for a specific user to generate a personalized half-life which accounts for the user's activity metrics such as amount of sleep, activity levels, etc. The system may use prior data previously recorded by the unit (or personal electronic device or computer). Additionally and/or alternatively, prior data may be imported from a third party health tracking platform such as a Fitbit® (Fitbit, Inc., San Francisco, Calif.), Apple® Health (Apple, Inc., Cupertino, Calif.), or another health tracking platform.

In any of the different variations described, any number of features between different variations are intended to be combined in any number of combinations. For instance, the unit (or personal device or computer) may be programmed to incorporate any of the TTG or Stay Green features with any of the cohort personalization variations and/or any other features described.

In one variation, an apparatus configured to encourage smoking cessation in a user may generally comprise a sampling unit configured to receive a sample breath from the user and determine a level of exhaled carbon monoxide from the sample breath as indicative of a carbon monoxide level within a body of the user, wherein the sampling unit is programmed to initiate a first timer for tracking a cumulative time upon a determination that the level of exhaled carbon monoxide is below a predetermined threshold, wherein the cumulative time corresponds to a first length of time in which subsequent sample breaths maintain the level of exhaled carbon monoxide below the predetermined threshold, and wherein the sampling unit is further programmed to initiate a second timer for tracking a countdown time upon a determination that the level of exhaled carbon monoxide is above the predetermined threshold, wherein the countdown time corresponds to a second length of time until the carbon monoxide level within the body of the user is expected to dissipate below the predetermined threshold.

In one variation of a method of encouraging smoking cessation in a user, the method may generally comprise receiving a sample breath from the user, and determining a level of exhaled carbon monoxide from the sample breath as indicative of a carbon monoxide level within a body of the user. A first timer may be initiated for tracking a cumulative time upon a determination that the level of exhaled carbon monoxide is below a predetermined threshold, wherein the cumulative time corresponds to a first length of time in which subsequent sample breaths maintain the level of exhaled carbon monoxide below the predetermined threshold. A second timer may be initiated for tracking a countdown time upon a determination that the level of exhaled carbon monoxide is above the predetermined threshold, wherein the countdown time corresponds to a second length of time until the carbon monoxide level within the body of the user is expected to dissipate below the predetermined threshold.

In one variation of a method of encouraging smoking cessation in a user, the method may generally comprise receiving a sample breath from the user and determining a level of exhaled carbon monoxide from the sample breath as indicative of a carbon monoxide level within a body of the user. The level of exhaled carbon monoxide may be visually displayed to the user and a first timer may be initiated for tracking a cumulative time upon a determination that the level of exhaled carbon monoxide is below a predetermined threshold. The cumulative time may be visually displayed to the user, wherein the cumulative time corresponds to a first length of time in which subsequent sample breaths maintain the level of exhaled carbon monoxide below the predetermined threshold and a second timer may be initiated for tracking a countdown time upon a determination that the level of exhaled carbon monoxide is above the predetermined threshold. The countdown time may be displayed to the user, wherein the countdown time corresponds to a second length of time until the carbon monoxide level within the body of the user is expected to dissipate below the predetermined threshold.

### BRIEF DESCRIPTION OF THE DRAWINGS

FIG. 1 illustrates a variation of a system which is able to receive the exhaled breath from a subject and detect various parameters and which can communicate with a one or more remote devices.

FIGS. 2A and 2B illustrate a variation of the personal sampling unit showing examples of eCO information for display to the user.

FIG. 3 illustrates a flow diagram of one variation of how the system may be programmed to implement the TTG and Stay Green strategies.

FIG. 4A illustrates examples of graphical user interfaces which may be displayed upon the screen of the unit to show the calculated PPM value displayed numerically as well as on a scale in a Stay Green mode.

FIG. 4B illustrates examples of graphical user interfaces which may be displayed upon the screen of the unit to show the calculated PPM value displayed numerically as well as on a scale in a TTG mode.

FIG. 5 illustrates a flow diagram of one variation of how the system may be programmed to account for smoking activities of the user.

### DETAILED DESCRIPTION OF THE INVENTION

The eCO values obtained from the user may be used to educate and motivate the user to quit by helping the user visualize and internalize the link between their smoking behavior and their measured eCO value. With the eCO value quantified, the half-life of CO within the user's body may be used to predict the amount of time for the CO to dissipate to a desirable level, e.g., 6 PPM or less which is the typical CO level of a non-smoking individual, and this information may be used to provide motivational content or guidance to the user. For instance, the targeted level of 6 PPM or less may be presented to the user as a “green” level where the estimated time until the CO is dissipated so that the detected amount is within the green level can be considered “Time To Green” (TTG).

The half-life of CO within a user's body may be used to monitor their response to cigarettes as a proxy for blood volume or hemoglobin measurement. The half-life value may also be used as an indicator for potential blood health issues such as low hemoglobin values.

Rather than using a standarized half-life value for CO dissipation within the body, an individualized model may be created for a specific user to generate a personalized half-life which accounts for a user's activity metrics such as amount of sleep, activity levels, etc. The model may also consider the number of cigarettes smoked as self-reported by the user for the personalized model in addition to the incorporated CO which may already be present within the user's body.

In obtaining the eCO from the user, certain biometric data of the user may be obtained by non-invasively detecting and quantifying the smoking behavior for a user based on measuring one or more of the user's biometric data; however, other biometric data can also be used. Such measurements or data collection can use a portable measuring unit or a fixed measuring unit, either of which communicates with one or more electronic devices for performing the quantification analysis. Alternatively, the analysis can be performed in the portable/fixed unit. For example, the portable unit can be coupled to a keychain, to the individual's cigarette lighter, cell phone, or other item that will be with the individual on a regular basis. Alternatively, the portable unit can be a stand-alone unit or can be worn by the individual.

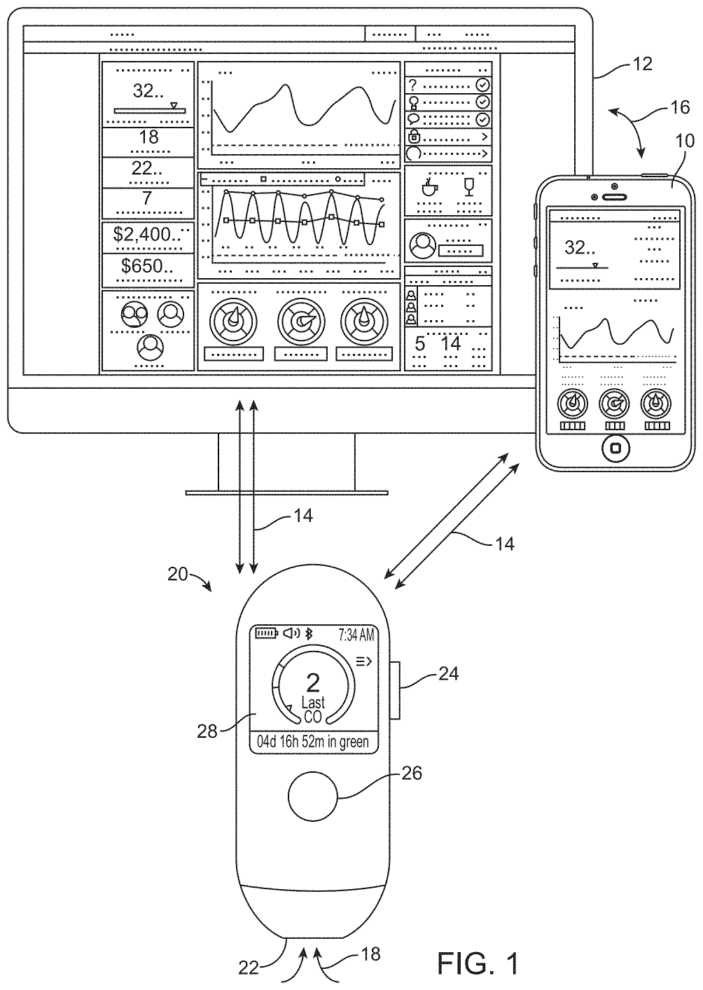

FIG. 1 illustrates one variation of a system and/or method in which a plurality of samples of biometric data are obtained from the user and analyzed to quantify the user's exposure to cigarette smoke such that the quantified information can be relayed to the individual, a medical caregiver, and/or other parties having a stake in the individual's health. The example discussed below employs a portable device 20 that obtains a plurality of samples of exhaled air from the individual with commonly available sensors that measure an amount of eCO within the sample of exhaled air. However, the quantification and information transfer are not limited to exposure of smoking based on exhaled air. As noted above, there are many sampling mechanisms to obtain a user's smoking exposure. The methods and devices described in the present example can be combined or supplemented with any number of different sampling mechanisms where possible while still remaining within the scope of the invention.

The measurement of eCO level has been known to serve as an immediate, non-invasive method of assessing a smoking status of an individual. The eCO levels for non-smokers can range between, e.g., 0 ppm to 6 ppm

As shown, a portable or personal sampling unit 20 may communicate with either a personal electronic device 10 or a computer 12. Where the personal electronic device 10 includes, but is not limited to a smartphone, cellular phone, or other personal transmitting device designed or programmed for receiving data from the personal sampling unit 20. Likewise, the computer 12 is intended to include a personal computer, local server, remote server, etc. Data transmission 14 from the personal sampling unit 20 can occur to both or either the personal electronic device 10 and/or the computer 12. Furthermore, synchronization 16 between the personal electronic device 10 and the computer 12 is optional. Either the personal electronic device 10, the computer 12, and/or the personal sampling unit 20 can transmit data to a remote server for data analysis as described herein. Alternatively, data analysis can occur, fully or partially, via a processor contained in a local device such as the sampling unit 20 (or the computer 12 or personal electronic device 10). In any case, the personal electronic device 10 and/or computer 12 can provide information to the individual, caretaker, or other individual as shown in FIG. 1.

The personal sampling unit 20 receives a sample of exhaled air 18 from the individual via a collection entry or opening 22. Hardware within the personal sampling unit 20 may include any variety of electrochemical gas sensor that detects CO gas within the breath sample, transmission hardware that transmits data 14 (e.g., via Bluetooth®, cellular, or other radio waves to provide transmission of data). The transmitted data and associated measurements and quantification are then displayed on either (or both) a computer display 12 or a personal electronic device 10. Alternatively, or in combination, any of the information can be selectively displayed on the portable sampling unit 20.

The personal sampling unit 20 (or personal breathing unit) can also employ standard ports to allow direct-wired communication with the respective devices 10 and 12. In certain variations, the personal sampling unit 20 can also include memory storage, either detachable or built-in, such that the memory permits recording of data and separate transmission of data. Alternatively, the personal sampling unit can allow simultaneous storage and transmission of data. Additional variations of the device 20 do not require memory storage. In addition, the unit 20 can employ any number of GPS components, inertial sensors (to track movement), and/or other sensors that provide additional information regarding the patient's behavior.

The personal sampling unit 20 can also include any number of input trigger (such as a switch or sensors) 24, 26. As described below, the input trigger 24, 26 may allow the individual to prime the device 20 for delivery of a breath sample 18 or to record other information regarding the cigarette such as quantity of cigarette smoked, the intensity, etc. In addition, variations of the personal sampling unit 20 may also associate a timestamp of any inputs to the device 20. For example, the personal sampling unit 20 can associate the time at which the sample is provided and provide the measured or inputted data along with the time of the measurement when transmitting data 14. Alternatively, the personal sampling device 20 can use alternate mechanisms to identify the time that the sample is obtained. For example, given a series of samples rather than recording a timestamp for each sample, the time periods between each of the samples in the series can be recorded. Therefore, identification of a timestamp of any one sample allows determination of the time stamp for each of the samples in the series.

In certain variations, the personal sampling unit 20 may be designed such that it has a minimal profile and can be easily carried by the individual with minimal effort. Therefore the input triggers 24 can comprise low profile tactile switches, optical switches, capacitive touch switches, or any commonly used switch or sensor. The portable sampling unit 20 can also provide feedback or information to the user using any number of commonly known techniques. For example, as shown, the portable sampling unit 20 can include a screen 28 that shows select information as discussed below. Alternatively or additionally, the feedback can be in the form of a vibrational element, an audible element, and a visual element (e.g., an illumination source of one or more colors). Any of the feedback components can be configured to provide an alarm to the individual, which can serve as a reminder to provide a sample and/or to provide feedback related to the measurement of smoking behavior. In addition, the feedback components can provide an alert to the individual on a repeating basis in an effort to remind the individual to provide periodic samples of exhaled air to extend the period of time for which the system captures biometric (such as eCO, CO levels, H₂ etc.) and other behavioral data (such as location either entered manually or via a GPS component coupled to the unit, number of cigarettes, or other triggers). In certain cases, the reminders can be triggered at higher frequency during the initial program or data capture. Once sufficient data is obtained, the reminder frequency can be reduced.

In obtaining the breath sample with the sampling unit 20, instructions may be provided on the personal electronic device 10 or computer display 12 for display to the subject in a guided breath test for training the subject to use the unit 20. Generally, the subject may be instructed, e.g., on the screen 28 of the electronic device 10, to first inhale away from the unit 20 and then to exhale into the unit 20 for a set period of time. The unit 20 may optionally incorporate one or more pressure sensors fluidly coupled with, e.g., check valves, to detect if the subject inhales through the unit 20.

Further examples of breath sampling devices and methods for determining and quantifying eCO levels from a user are described in further detail in various patents, e.g., U.S. Pat. Nos. 9,861,126; 10,206,572; 10,306,922; and 10,335,032, each of which is incorporated herein by reference in its entirety and for any purpose.

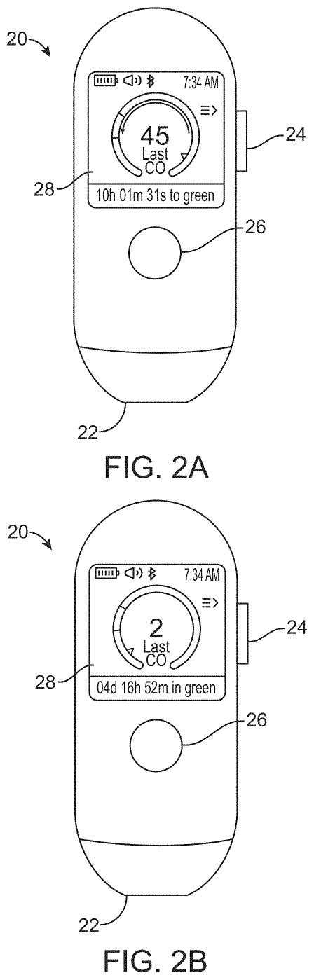

FIG. 2A shows a front view of the unit 20 with an example of a display presented upon the screen 28 once a sample of the user's breath has been received within the unit 20 and processed. Once the system has determined the level of CO present within the sample, the unit 20 may display the measured CO level as well as a visual indication of where the measured level rates upon a scale given that a user's CO level may be used as an indicator to determine their smoking status. FIG. 2A illustrates an example where the eCO is measured to have a level of 45 PPM and further displays a timer indicating the length of time that should pass before the CO level within the user's body will drop into the green level when the CO level is between 0 to 6 PPM in the user's body (“Time To Green” or TTG), provided that the user does not smoke again in the interim. This example shows that for the measured eCO level of 45 PPM, a TTG of several hours should pass before the CO levels within the user is expected to drop into the green level (e.g., 0 to 6 PPM), provided the user does not smoke during this period.

Furthermore, because eCO is typically well-correlated with blood CO content (CO-bound hemoglobin or carboxyhemoglobin), an increase in eCO may represent an increase in the percentage of CO-bound hemoglobin. This is also representable to the user as the total amount of CO-bound hemoglobin divided by the percentage of total hemoglobin.

Presented with this information, the user may be motivated to set a goal to reach the green level which represents the typical CO level of a non-smoking individual. Each time the user takes a measurement of eCO with the unit 20 (or personal electronic device 10 or computer 12) may calculate the approximate TTG until the user will reach the green level. In other alternative variations, the user can also set user-defined goals other than getting to green (TTG). For example, if the user has never recorded a value under, e.g., 15 PPM, the user may set a goal of under, e.g., 12 PPM, so the user-defined goal is more readily achievable and the user may become more motivated for attaining this alternative goal.

In obtaining the breath samples from the user, it may be desirable to obtain breath samples which are generally consistent with the user's normal activities since the eCO readings may vary according to the time of day or the user's smoking schedule throughout the day. Hence, it may be useful to compare eCO readings over time that are obtained at specific times or in specific time ranges which are consistent between days such that a model of daily CO levels may be based on a cigarette schedule and/or past CO levels. This may also allow the user to more readily respond to changes, for instance, where a user can experiment with eliminating specific cigarettes from their daily schedule to investigate how this would affect their overall CO levels. Furthermore, this may also allow for the user to more readily respond to deviations from the model as well, for instance, when a CO value is different from a model for a given particular time or number of cigarettes logged.

In one variation, the user may be prompted or alerted, e.g., via the unit or personal device, to provide a specified number of breath samples at specified (or approximate) times of the day (e.g., four samples per day). Examples of times may include, e.g., upon waking, before lunch, mid-afternoon, and prior to going to bed. Given sufficient data for each of these times, the eCO readings at these specified times may be charted over the course of a period of time (such as over a week) to provide a better and cleaner visual indication of progress and opportunity for insight to the user. This approach may also provide a relatively better overview of progress to the user (and better motivation) over simply providing a visual chart at every eCO reading taken over the period of time.

In addition to specified times or time ranges, other parameters of interest may be used to produce a simplified visual indicator of progress. Such parameters may include, e.g., minimum, maximum, and/or average eCO values. Other additional parameters may also include, e.g., quartile range such as the average of the lowest quartile of samples provided on a given day. When these parameters are combined with a reasonably accurate cigarette log or count (as described in further detail below), additional inferences or automatic categorization of samples may be made, e.g., obtaining a reading before the first cigarette of the day or obtaining a reading after the first cigarette of the day.

In one variation, the unit 20 may use a standardized CO half-life time (e.g., around 4 hours) in determining the TTG of the user. This standardized CO half-life time may be based upon a conservative population-average in which the CO half-life time (e.g., the time for a CO level to dissipate to half within a body) is an average value obtained from a population. To account for variables such as sensor variability, sensor noise, natural breath readings, etc. the start and end values may be compensated as appropriate, e.g., to target a value of 4 PPM instead of a value of 6 PPM which is at the end of the green range.

In another variation, an individualized model may be created for a specific user to generate a personalized half-life which accounts for the user's activity metrics such as amount of sleep, activity levels, etc. The system may use prior data previously recorded by the unit 20 (or personal electronic device 10 or computer 12). Additionally and/or alternatively, prior data may be imported from a third party health tracking platform such as a Fitbit® (Fitbit, Inc., San Francisco, Calif.), Apple® Health (Apple, Inc., Cupertino, Calif.), or another health tracking platform.

FIG. 2B shows the unit 20 with another variation presented upon the screen 28 in which the user may provide a measured eCO value which is within the green level (e.g., 0 to 6 PPM). This indication lets the user know that enough time has passed (TTG) that the CO levels within their body has dissipated sufficiently and further indicates that the user has refrained from smoking long enough to reach the green level. In other words, the user has succeeded in the challenge to refrain from smoking long enough to have arrived at their goal.

However, some users may consider their reaching this goal as a license to then smoke as a “reward”. To prevent misperception or inappropriate use of the TTG goal, the system may further incorporate an additional strategy of encouraging the user to maintain their eCO levels within the green level for as long as possible once they have reached it (“Stay Green”). For example, after the first instance of an eCO level falling within the green level, the Stay Green timer may start counting up from zero and the unit 20 may display the time that the user has stayed or maintained their eCO within the green level. This displayed “Stay Green” time may encourage the user to compare their “Stay Green” time against previous “Stay Green” times in an attempt to try and increase their longest “Stay Green” streak over time. At subsequent samples maintained within the green level, the Stay Green time may be locked in and continue showing the cumulative time within the green level. Even after quitting, the user may enjoy or obtain satisfaction from continually increasing their green streak.

During this “Stay Green” phase, the system may prompt the user to sample regularly in order to continue accumulating time in the “Stay Green” phase. For instance, the user may be encouraged or required to sample at predetermined time periods, e.g., every 4 hours or less, during waking hours to increase their “Stay Green” time or to add the full intervening time to the streak. If a subsequent sample is measured to fall above the green level, the Stay Green time will be stopped and the TTG timer may begin as further motivation to return to the green level.

To account for natural variability in rate of exhausting CO, and accuracy of the device, it is desirable to give a conservative estimate of the TTG value, e.g., a value which is slightly longer than the standard half-life calculation with an average half-life value would give. This is especially true if the current eCO value is closer to the limit of 6 PPM.

On the other hand, simply using a higher half-life value (e.g., 5 hours) may exaggerate the TTG values for higher CO levels and may be unnecessarily discouraging to the user. Therefore, a pair of equations may be used at values closer or farther from the 6 PPM limit value, with the cutover point selected to maintain continuity between the two equations (e.g., both equations give the same value at the cutover point). However the equations and cutover point given are one example of any number of equations or parameters that could be used. In the case where we can collect enough data from the person to personalize their half-life and also collect, for example, activity information, a more accurate TTG time based on their personal characteristics may be provided. Hence, each time a non-green value is detected, the new TTG end-point time is calculated using one of two approaches. For PPM values which are equal to or greater than 12 PPM, the Time to Green (TTG₁) may be calculated using the following equation (1):

$$T T {G}_{1} = 1 + 4 [ \frac{ln ( \frac{{CO}_{ppm}}{6} )}{ln ( 2 )} ] \qquad ( 1 )$$

where,
TTG₁=Time to Green (in hours)

### COₚₚₘ=PPM Reading (≥12 PPM)

For PPM values which are less than 12 PPM, the Time to Green (TTG₂) may calculated using the following equation (2):

$$T T {G}_{2} = 5 [ \frac{ln ( \frac{{CO}_{ppm}}{6} )}{ln ( 2 )} ] \qquad ( 2 )$$

where,
TTG₂=Time to Green (in hours)

### COₚₚₘ=PPM Reading (<12 PPM)

Additionally and/or alternatively, a standardized table may also be utilized in determining the approximate TTG based on a particular PPM reading obtained from a sample, as shown in the following Table 1:

TABLE 1
Time To Green Based on PPM Readings

| | Time To Green | Time To Green |
|---|---|---|
| PPM Reading | (Hours) | (Hours-Mins) |
| 7 | 1.1 | 1 h 06 m |
| 8 | 2.1 | 2 h 04 m |
| 9 | 2.9 | 2 h 55 m |
| 10 | 3.7 | 3 h 41 m |
| 12 | 5.0 | 5 h 00 m |
| 14 | 5.9 | 5 h 53 m |
| 16 | 6.7 | 6 h 39 m |
| 18 | 7.3 | 7 h 20 m |
| 20 | 7.9 | 7 h 56 m |
| 25 | 9.2 | 9 h 14 m |
| 30 | 10.3 | 10 h 17 m |
| 35 | 11.2 | 11 h 10 m |
| 40 | 11.9 | 11 h 56 m |
| 45 | 12.6 | 12 h 37 m |
| 50 | 13.2 | 13 h 14 m |
| 55 | 13.8 | 13 h 47 m |
| 60 | 14.3 | 14 h 17 m |
| 65 | 14.7 | 14 h 44 m |
| 70 | 15.2 | 15 h 10 m |
| 75 | 15.6 | 15 h 34 m |
| 80 | 15.9 | 15 h 56 m |
| 85 | 16.3 | 16 h 17 m |
| 90 | 16.6 | 16 h 37 m |
| 95 | 16.9 | 16 h 56 m |
| 100 | 17.2 | 17 h 14 m |

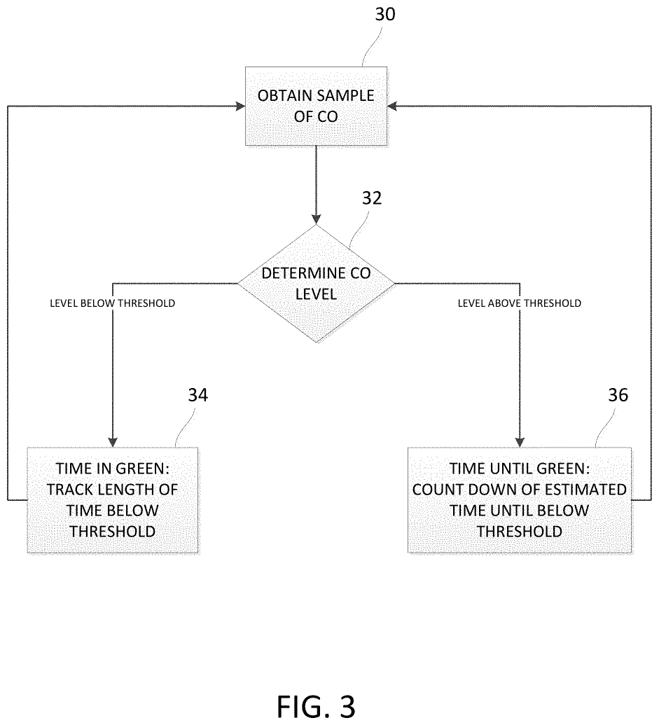

FIG. 3 illustrates a flow diagram of one variation of how the system may be programmed to implement the TTG and Stay Green strategies with respect to the unit 20. Once the eCO sample is obtained from the user 30, the processor either within the unit 20, personal electronic device 10, or computer 12 may be programmed to determine the corresponding CO level within the sample 32. If the resulting PPM level is below a predetermined threshold value (e.g., 6 PPM), the system may determine the time in the green level to track the cumulative length of time below the threshold level 34 for display to the user (Stay Green). However, if the sample CO level is above the predetermined threshold value, the system may then determine the time until the green level is reached 36 by starting (or continuing) a count down of the estimated time until the CO level is expected to fall below the threshold value within the user's body (TTG). Each time the user provides a sample to the unit 20 for eCO determination, the calculation of either TTG or cumulative time until green (Stay Green) may be determined. If the user smokes or inhales cigarette smoke in-between samples, the TTG or Stay Green values are adjusted accordingly.

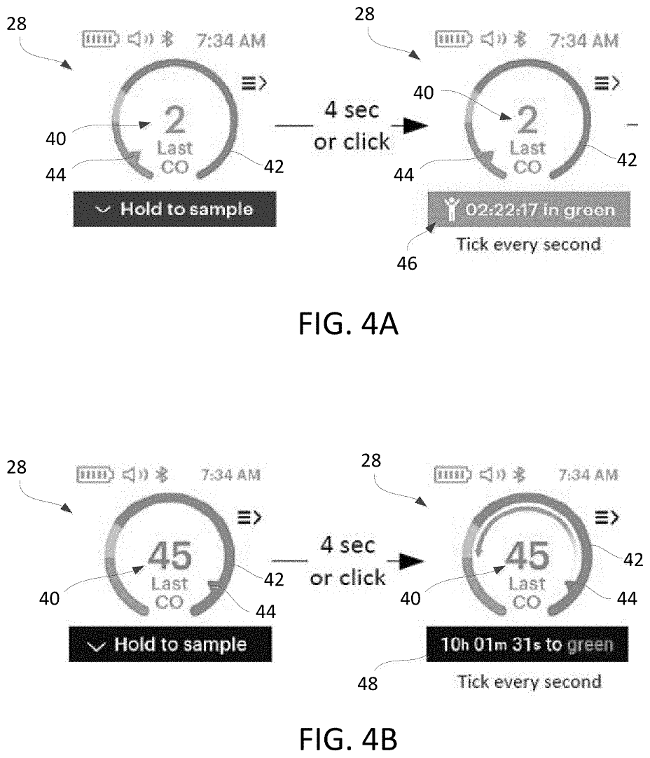

An example of the graphical user interface which may be displayed upon the screen 28 of the unit 20 is illustrated in FIG. 4A which shows the calculated PPM value 40 displayed numerically as well as on a scale 42 which is delineated into a green zone (e.g., 0 to 6 PPM), yellow zone (e.g., >6 to 9 PPM), and red zone (e.g., >9 PPM) which are designed to provide the user a relative scale of how their eCO level compares. (For sample readings which fall within the yellow zone, any previously continuing Stay Green counters may continue to accumulate time but a new TTG value may be displayed as well.) A visual marker 44 may move along the scale 42 depending on where the user's eCO level resides. In this example, a measured eCO value of 2 PPM shows the visual marker 44 located along the scale 42 within the green zone. A corresponding timer 46 may be accordingly displayed showing the cumulative length of time that the user has managed to maintain their eCO levels within the green zone (Stay Green) as an encouragement and challenge to the user to continue keeping their eCO levels within the green zone by continuing to refrain from smoking activities.

In the event that the user's eCO level is measured at a higher level, e.g., 45 PPM as shown in the example illustrated in FIG. 4B, the corresponding PPM value 40 may be displayed upon the scale 42 but with the visual marker 44 located within the red zone of the scale 42 to reflect the elevated reading. The screen 28 may then display the count down timer 48 until the CO levels within the user are estimated to drop into the green zone (TTG) as a motivation to the user to further refrain from smoking activities.

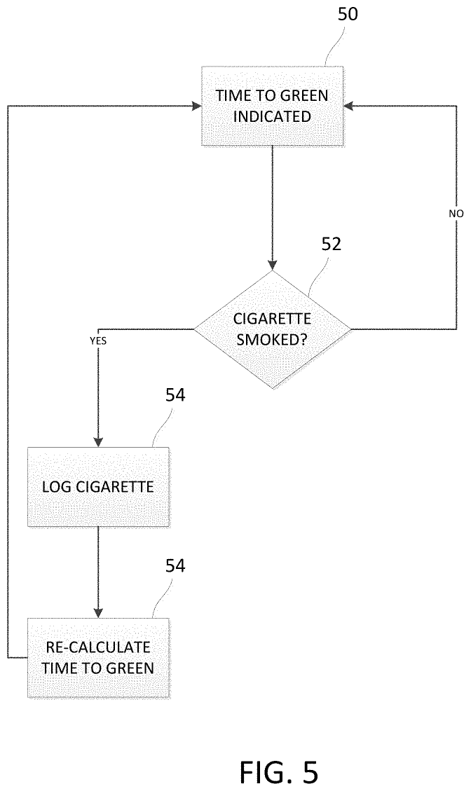

FIG. 5 illustrates a flow diagram of one variation of how the system may be programmed to account for smoking activities of the user, e.g., in-between the time when their breath samples are taken by the unit 20. If the user were to smoke prior to taking a breath sample with the unit 20, the TTG time would be calculated and indicated 50 to the user who may then be optionally questioned as to whether they had smoked 52 and then also provided the option to log the number of cigarettes 54 they had smoked into the unit 20. The unit 20 or system may then re-calculate the time to green 54 so that an updated TTG time may be calculated and/or displayed to the user.

As yet another variation for providing motivation and context to the user, cohort benchmarking information may be provided for display to the user in comparing the user's status or progress against that of similar users. Cohorts of other similar users may be constructed using various criteria to produce comparisons that may be helpful and motivation to the user. Some examples of various criteria which may be used for categorizing similar user cohorts may include, for instance, being of the same age, belonging to the same sign-up cohort (e.g., the same company or worksite, etc.), having similar initial smoking profiles (e.g., smoking half pack per day, etc.), same sex, similar weight, etc.

Status and progress indicators may also be chosen and presented to the user in a manner that is motivational to the user, for example, rather than providing a message such as “Your CO values this week are 0.2% higher than others from your city”, the indicator may instead be provided in a motivational matter such as “You've decreased your average weekly CO reading by 15%, which puts it 10% lower than others who started the program at the same time!”

Alternatively, rather than comparing the indicator of interest within a defined cohort, a cohort can be constructed using the indicator of interest to provide a helpful or motivational message to the user based on that cohort's performance. For example, “You've decreased your average evening CO by 20% week-over-week! Others who have done this have been 40% more likely to have a successful quit in the next month.”

Because CO readings are affected by more than smoking behavior alone (e.g., body weight), modified metrics may also be used to “normalize” the data for comparison purposes. Percentage decrease is one example while another example may include excess CO where the value over a standard baseline (e.g., 6 PPM) or the value over a personal baseline as generated from previous data. In the case where the value is presented relative to a personal baseline, other alternative parameters may be presented such as the amount of time spent with the user's CO level under 50% of a previous week's average or under a previous maximum level. For instance, a message may be presented such as “90% of your CO readings this week were below last week's average! Only 10% of users are able to achieve that in the first 4 weeks!”

In any of the different variations described, any number of features between different variations are intended to be combined in any number of combinations. For instance, the unit 20 (or personal device or computer) may be programmed to incorporate any of the TTG or Stay Green features with any of the cohort personalization variations and/or any other features described.

While illustrative examples are described above, it will be apparent to one skilled in the art that various changes and modifications may be made therein. Moreover, various apparatus or procedures described above are also intended to be utilized in combination with one another, as practicable. The appended claims are intended to cover all such changes and modifications that fall within the true spirit and scope of the invention.

What is claimed is:

1. An apparatus configured to encourage smoking cessation in a user, comprising:
a sampling unit configured to receive a sample breath from the user and determine a level of exhaled carbon monoxide from the sample breath as indicative of a carbon monoxide level within a body of the user,
wherein the sampling unit is programmed to initiate a first timer for tracking a countdown time upon a determination that the level of exhaled carbon monoxide is above a predetermined threshold, and
wherein the countdown time corresponds to a first length of time until the carbon monoxide level within the body of the user is expected to dissipate below the predetermined threshold.

2. The apparatus of claim 1 wherein the sampling unit is further programmed to initiate a second timer for tracking a cumulative time upon a determination that the level of exhaled carbon monoxide is below the predetermined threshold,
wherein the cumulative time corresponds to a second length of time in which subsequent sample breaths maintain the level of exhaled carbon monoxide below the predetermined threshold.

3. The apparatus of claim 1 wherein the sampling unit comprises a processor for determining the level of exhaled carbon monoxide.

4. The apparatus of claim 1 wherein the sampling unit comprises a screen for displaying the level of exhaled carbon monoxide to the user.

5. The apparatus of claim 1 wherein the sampling unit is further programmed to display a visual indicator of the cumulative time.

6. The apparatus of claim 1 wherein the sampling unit is further programmed to display a visual indicator of the countdown time.

7. The apparatus of claim 1 wherein the predetermined threshold comprises a carbon monoxide level of 6 PPM.

8. A method of encouraging smoking cessation in a user, comprising:
receiving a sample breath from the user;
determining a level of exhaled carbon monoxide from the sample breath as indicative of a carbon monoxide level within a body of the user; and
initiating a first timer for tracking a countdown time upon a determination that the level of exhaled carbon monoxide is above a predetermined threshold, wherein the countdown time corresponds to a first length of time until the carbon monoxide level within the body of the user is expected to dissipate below the predetermined threshold.

9. The method of claim 8 further comprising initiating a second timer for tracking a cumulative time upon a determination that the level of exhaled carbon monoxide is below the predetermined threshold, wherein the cumulative time corresponds to a second length of time in which subsequent sample breaths maintain the level of exhaled carbon monoxide below the predetermined threshold.

10. The method of claim 8 wherein receiving the sample breath comprises receiving the sample breath into a sampling unit.

11. The method of claim 8 wherein determining the level of exhaled carbon monoxide further comprises visually displaying the level to the user.

12. The method of claim 9 wherein initiating the second timer further comprises displaying the cumulative time to the user until at least one of the subsequent sample breaths result in exhaled carbon monoxide above the predetermined threshold.

13. The method of claim 8 further comprising adjusting the countdown timer upon receiving a subsequent sample breath having a second level of exhaled carbon monoxide which is higher than the level of exhaled carbon monoxide.

14. The method of claim 8 wherein initiating the first timer further comprises determining the countdown time corresponding to a half-life value of the carbon monoxide level within the body of the user.

15. The method of claim 8 wherein the predetermined threshold comprises a level of 6 PPM.

16. A method of encouraging smoking cessation in a user, comprising:
receiving a sample breath from the user;
determining a level of exhaled carbon monoxide from the sample breath as indicative of a carbon monoxide level within a body of the user;
visually displaying the level of exhaled carbon monoxide to the user;
initiating a first timer for tracking a cumulative time upon a determination that the level of exhaled carbon monoxide is below a predetermined threshold;
displaying the cumulative time to the user, wherein the cumulative time corresponds to a first length of time in which subsequent sample breaths maintain the level of exhaled carbon monoxide below the predetermined threshold; and
initiating a second timer for tracking a countdown time upon a determination that the level of exhaled carbon monoxide is above the predetermined threshold; and
displaying the countdown time to the user, wherein the countdown time corresponds to a second length of time until the carbon monoxide level within the body of the user is expected to dissipate below the predetermined threshold.

17. The method of claim 16 wherein receiving the sample breath comprises receiving the sample breath into a sampling unit.

18. The method of claim 16 wherein determining the level of exhaled carbon monoxide further comprises visually displaying the level to the user.

19. The method of claim 16 wherein initiating the first timer further comprises displaying the cumulative time to the user until at least one of the subsequent sample breaths result in exhaled carbon monoxide above the predetermined threshold.

20. The method of claim 16 further comprising adjusting the countdown timer upon receiving a subsequent sample breath having a second level of exhaled carbon monoxide which is higher than the level of exhaled carbon monoxide.

21. The method of claim 16 wherein initiating the second timer further comprises determining a half-life value of the carbon monoxide level within the body of the user.
